(19) United States
(12) Patent Application Publication
(10) Pub. No.: US 2005/0215884 A1
(43) Pub. Date: Sep. 29, 2005

(54) Evaluation of Alzheimer's disease using an independent component analysis of an individual's resting-state functional MRI

(76) Inventors: Michael D. Greicius, Palo Alto, CA (US); Vinod Menon, Los Altos, CA (US); Allan L. Reiss, Stanford, CA (US)

Correspondence Address: LUMEN INTELLECTUAL PROPERTY SERVICES, INC., 2345 YALE STREET, 2ND FLOOR, PALO ALTO, CA, 94306, US

(21) Appl. No.: 11/067,612

(22) Filed: Feb. 25, 2005

Related U.S. Application Data
(60) Provisional application No. 60/548,306, filed on Feb. 27, 2004.

(51) Int. Cl. A61B 5/05

(52) U.S. Cl. 600/410

(57) ABSTRACT

A clinically valuable method is provided for evaluating the onset or progression of Alzheimer's disease using a non-invasive biomarker obtained from an independent component analysis (ICA) of an individual's resting state functional MRI. The method is relatively more automated and objective than previous methods and exploits dysfunctional connectivity across an entire network of brain regions in Alzheimer's disease. It eliminates the need for investigator's intervention as much as possible and is more robust than structural and functional methods targeting the hippocampus.

### STATEMENT REGARDING FEDERALLY SPONSORED RESEARCH OR DEVELOPMENT

The present invention was supported in part by grant numbers MH19938 and HD40761 both from the National Institutes of Health (NIH/NCI). The U.S. Government has certain rights in the invention.

### FIELD OF THE INVENTION

The present invention relates generally to the field of Alzheimer's Disease. More particularly, the present invention relates to methods of detecting and evaluating Alzheimer's Disease, at various stages, in individual subjects.

### BACKGROUND

At present, Alzheimer's disease is unpreventable and incurable, severely limiting physical and mental abilities and devastating memory function in about four million people in the U.S. Given the demographics of an aging population and barring significant breakthroughs in diagnosis and treatment, it is estimated that as many as 14 million people will suffer from the brain disorder by the year 2050. However, advances in drug development and other interventions are starting to show better results in delaying the onset of the Alzheimer's and in treating symptoms. For both current and future therapy options, the ability to accurately measure pre-clinical risk for individual patients may enable physicians to intervene with good effect before the damage cannot be reversed.

Several methods have been proposed to identify signs and establish biologic markers of the preclinical phase of Alzheimer's disease using e.g. genetic markers, plasma concentrations, or hippocampus atrophy measurements via magnetic resonance imaging (MRI). Li et al. evaluated a clinical marker obtained by analyzing resting-state functional MRI (fMRI) of a small, isolated region of interest of a subject's brain (Li et al. (2002) in a paper entitled “Alzheimer Disease: Evaluation of a Functional MR Imaging Index as a Marker” and published in Radiology 225:253-259). Li's specific region of interest was the hippocampus from which they measured cross-correlation coefficients of spontaneous low frequency components between possible pairs of voxel time courses in the brain region. A problem with Li's approach is the need of an investigator's intervention for verification of the region of interest. Such verification is difficult to standardize and makes comparing test results among subjects in a preclinical phase and during intervention non-trivial. In addition, Li's approach is restricted to the hippocampus and does not take advantage of the broader scope of brain pathology in Alzheimer's disease (e.g. posterior cingulate cortex, temporo-parietal regions, etc.). Accordingly, to develop a robust, clinically valuable biomarker for Alzheimer's disease, it is considered an advance in the art to develop new, relatively more automated and objective methods that exploit dysfunctional connectivity across an entire network of brain regions in Alzheimer's disease. Preferably such methods would eliminate the need for investigator's intervention as much as possible and prove more robust than structural and functional methods targeting the hippocampus.

### SUMMARY OF THE INVENTION

The present invention is a method of evaluating the onset or progression of Alzheimer's disease using a non-invasive clinical marker obtained from an independent component analysis (ICA) of an individual's resting state functional MRI. ICA components of the individual's resting state functional MRI are matched with a reference template representing a default-mode network of subjects (e.g. healthy subjects). For each of the matched components a goodness-of-fit score is assigned after which the component with the highest score is selected as the default-mode network component for the individual's resting state functional MRI. The non-invasive clinical marker is determined by comparing the score of this default-mode network component with reference values. It is this non-invasive clinical marker that is used to evaluate the onset or progression of Alzheimer's disease in the individual.

The method provides a clinically valuable biomarker for Alzheimer's disease. Furthermore, it is a relatively more automated and objective method compared to previous methods and exploits dysfunctional connectivity across an entire network of brain regions in Alzheimer's disease. It eliminates the need for investigator's intervention as much as possible and is more robust than structural and functional methods targeting the hippocampus.

### BRIEF DESCRIPTION OF THE FIGURES

The present invention together with its objectives and advantages will be understood by reading the previous summary and following description in conjunction with the drawings, in which:

FIG. 1 shows axial images of a default-mode network as detected with the ICA-based approach of the present invention in a group of healthy young adults. The arrow indicates the posterior cingulate cortex. The left side of the image corresponds to the left side of the brain. The numbers beneath each image refer to the z-coordinate in Talairach space. T-score bars are shown at right.

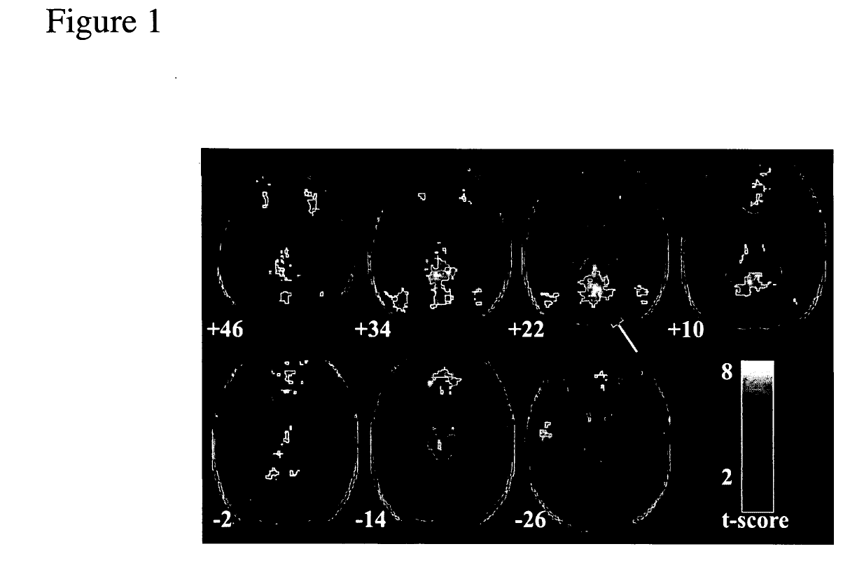

Functional images were overlayed on the group-averaged structural image. Joint height and extent thresholds of p<0.001 were used to determine significant clusters.

FIG. 2 shows axial images of a default-mode network for Alzheimer's patients. The top arrow indicates the posterior cingulate cortex. The left side of the image corresponds to the left side of the brain. The numbers beneath each image refer to the z-coordinate in Talairach space. T-score bars are shown at right.

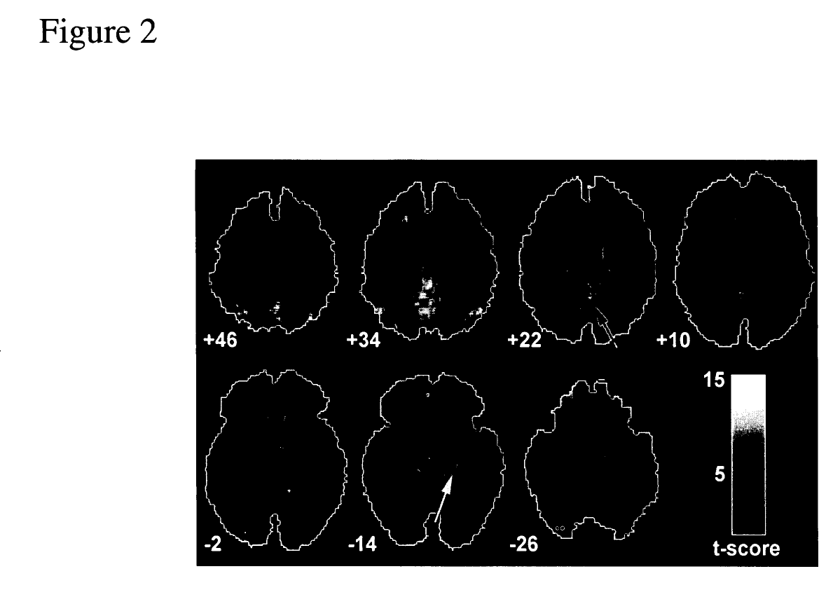

Functional images were overlayed on the group-averaged structural image. Joint height and extent thresholds of p<0.001 were used to determine significant clusters.

FIG. 3 shows a scattergram of the goodness-of-fit (to an ICA-derived default-mode template) for each subject in a group of patients with Alzheimer's disease (AD), a group of healthy elderly, and a group of patients with frontotemporal lobar degeneration (FTLD)—a non-AD dementia. An analysis of variance showed a main effect of diagnosis and post-hoc tests showed that Alzheimer's disease scores were significantly less than those of healthy controls and the FTLD group (p<0.05). The horizontal line indicates a cutoff point of 2.7 where this method correctly classified 8/9 Alzheimer's disease patients, 7/7 healthy controls, and 4/5 FTLD patients yielding a sensitivity of 89% in detecting Alzheimer's disease and 100% specificity in distinguishing Alzheimer's disease from healthy aging and 80% specificity in distinguishing Alzheimer's disease from FTLD.

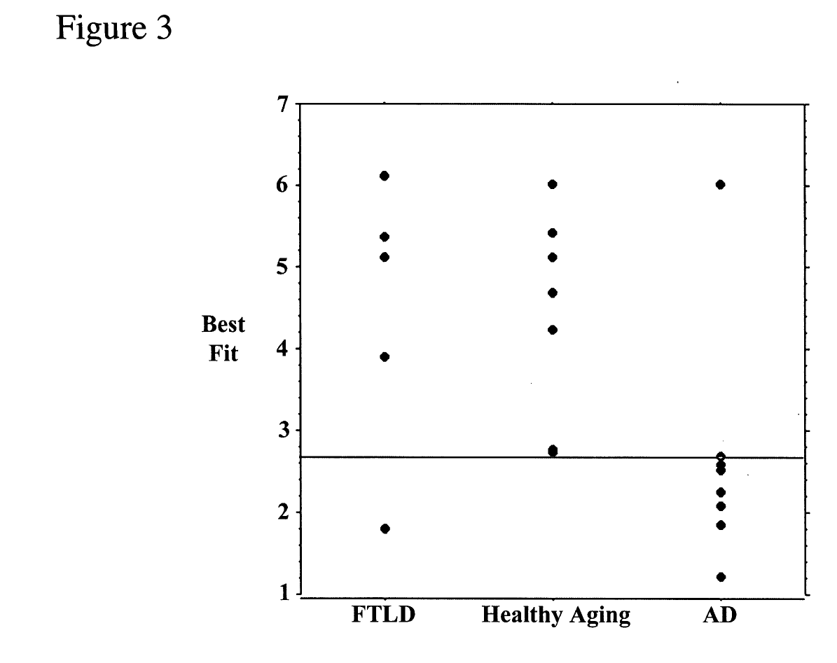

### DETAILED DESCRIPTION OF THE INVENTION

Acquiring Resting-State Data

Using a functional MRI (fMRI) protocol subjects are scanned during a standard period of rest. One may also acquire more than one resting-state scan and use the best score of several scans, the median score, the mean, or the like. Standardized instructions are given such as “for the next 6 minutes please relax and try not to move”.

Preprocessing Steps

Typical fMRI preprocessing steps are performed on the resting-state data, e.g. realignment, normalization, and/or smoothing. The normalization step may take place before or after the Independent Component Analysis (ICA) has been performed. However, normalization needs to be performed before the default-mode component is matched to the standard template (see below).

Independent Component Analysis (ICA)

ICA is a statistical technique that separates a set of signals, in this case fMRI data, into independent—uncorrelated and non-Gaussian—spatiotemporal components. The application of ICA in this invention will vary depending on the particular ICA approach and software used. Typically, in one embodiment, 180 or so time-points are concatenated into a single 4-dimensional image. Spatial ICA is performed on this image and some number, n, of independent components is generated. In a typical example, between 20 or 40 components would be generated and analyzed. If the data has not yet been normalized than it is normalized at this stage to the same space (e.g. Talairach space) defined for the standard template.

Automated Selection of the Default-Mode Component

The n components generated by ICA are then compared to the standard reference template. In this comparison a goodness-of-fit score is assigned to each component. The single component with the highest goodness-of-fit score is selected as the default-mode component.

The goodness-of-fit is obtained with a matching algorithm. In a publication by the inventors a nonlinear template-matching procedure was described that involved taking the average z-score of voxels falling within the template minus the average z-score of voxels outside the template and selecting the component in which this difference (the goodness-of-fit) was the greatest (Greicius et al. (2004) in a paper entitled “Default-mode network activity distinguishes Alzheimer's disease from healthy aging: Evidence from functional MRI” and published in PNAS 1011(13):4637-4642—this paper is hereby incorporated for all that it discloses). Z-scores here reflect the degree to which a given voxel's time-series correlates with the time-series corresponding to the specific ICA component (scaled by a residual noise estimate). Any number of alternative goodness-of-fit approaches can be adopted such as using a weighted template (reflecting regional differences in activity within the network) instead of a binary template or using voxel values other than the z-scores corrected for residual noise used in this example.

Goodness-of-Fit Metric

The score obtained, in the step above, for the default-mode component is the subject's goodness-of-fit score. This may be used by itself or one could obtain several scores from serial resting-state scans and use a statistical metric obtained from multiple scans (e.g. best goodness-of-fit score, mean or median goodness-of-fit score, or the like). A subject's goodness-of-fit metric is then compared to a previously acquired database with the range of goodness-of-fit scores (reference values) in normal subjects, subjects with non-Alzheimer's dementias, and subjects with Alzheimer's disease and the probability of the subject having—or in the case of patients with mild cognitive impairment, developing—Alzheimer's disease is determined.

Construction of the Template

The goodness-of-fit score reflects how well a given subject's default-mode network component matches a standard template of the network. Among several possibilities, this standard template may include an averaged map of the network in healthy young subjects, an averaged map of the network in healthy elderly subjects, or a difference map showing regions in the network where Alzheimer's disease patients show less activity than healthy elderly subjects. One could also derive a template showing regions within the default-mode network where Alzheimer's disease patients show less activity than healthy elderly patients.

Whichever template is decided upon, it is constructed by combining the default-mode components from, for example, 10 or more healthy young subjects, into a group-averaged map (e.g. using Statistical Parametric Mapping (SPM) to create a one-sample t-test map) or, as a second example, creating a map for each of two groups (healthy elderly and Alzheimer's disease) and creating a difference map (e.g. using SPM to create a two-sample t-test map). The template can be binary such that all voxels within it are equally weighted or it can reflect the weight of different regions within the network such that regions whose time-series are more tightly correlated with the average time-series of the component are weighted more strongly.

The present invention has now been described in accordance with several exemplary embodiments, which are intended to be illustrative in all aspects, rather than restrictive. Thus, the present invention is capable of many variations in detailed implementation, which may be derived from the description contained herein by a person of ordinary skill in the art. All such variations and other variations are considered to be within the scope and spirit of the present invention as defined by the following claims and their legal equivalents.

1. A method of evaluating the onset or progression of Alzheimer's disease using a non-invasive clinical marker obtained from an independent component analysis of an individual's resting state functional MRI, comprising the steps of:
(a) matching n components of said independent component analysis of said individual's resting state functional MRI with a reference template representing a default-mode network;
(b) assigning a goodness-of-fit score to said matched n components;
(c) selecting the component with the highest score from said goodness-of-fit scores as the default-mode network component for said individual's resting state functional MRI;
(d) determining said non-invasive clinical marker by comparing the score of said default-mode network component with reference values; and
(e) evaluating for said individual an onset or progression of Alzheimer's disease using said non-invasive clinical marker.

2. The method as set forth in claim 1, wherein said reference values are goodness-of-fit scores of default-mode networks in healthy or normal individuals, individuals with non-Alzheimer's dementias, individuals with Alzheimer's disease, or individuals with mild cognitive impairment.

3. The method as set forth in claim 1, wherein said non-invasive clinical marker reflects the probability of said individual having or developing Alzheimer's disease.

4. The method as set forth in claim 1, wherein said matching includes a nonlinear template-matching method, a linear template-matching method, a weighted template-matching method or a binary template-matching method.
